(19) United States
(12) Patent Application Publication
(10) Pub. No.: US 2011/0046473 A1
(43) Pub. Date: Feb. 24, 2011

(54) EEG TRIGGERED FMRI SIGNAL ACQUISITION

(75) Inventors: Anantha Pradeep, Berkeley, CA (US); Robert T. Knight, Berkeley, CA (US); Ramachandran Gurumoorthy, Berkeley, CA (US)

Correspondence Address: Weaver Austin Villeneuve & Sampson LLP, P.O. BOX 70250, OAKLAND, CA, 94612-0250, US

(73) Assignee: NeuroFocus, Inc., Berkeley, CA (US)

(21) Appl. No.: 12/544,934

(22) Filed: Aug. 20, 2009

(51) Int. Cl. A61B 5/055; A61B 5/0476

(52) U.S. Cl. 600/413; 600/544

(57) ABSTRACT

Neuro-response data including Electroencephalography (EEG) and Functional Magnetic Resonance Imaging (fMRI) data is collected, filtered and/or analyzed to evaluate the effectiveness of stimulus materials such as marketing and entertainment materials. A data collection mechanism obtains fMRI signals indicating a hemodynamic response to marketing or entertainment stimuli. In certain embodiments, such signals include region-specific blood oxygen level dependent (BOLD) signals that correlate with region-specific neural activity. fMRI signal acquisition is triggered by one or more EEG signatures indicating neural activity in response to exposure to stimulus materials.

### TECHNICAL FIELD

The present disclosure relates to performing audience response analysis using EEG and fMRI.

### DESCRIPTION OF RELATED ART

Conventional systems for determining the effectiveness of the stimulus material such as entertainment and marketing rely on either survey based evaluations or limited neurophysiological measurements used in isolation. These conventional systems provide some useful data but are highly inefficient and inaccurate due to a variety of semantic, syntactic, metaphorical, cultural, social, and interpretative errors and biases. The systems and techniques themselves used to obtain neurophysiological measurements are also highly limited.

Consequently, it is desirable to provide improved methods and apparatus for determining the effectiveness of stimulus material.

### BRIEF DESCRIPTION OF THE DRAWINGS

The disclosure may best be understood by reference to the following description taken in conjunction with the accompanying drawings, which illustrate particular example embodiments.

FIG. 1 illustrates one example of a system for determining the effectiveness of marketing and entertainment by using central nervous system measures, autonomic nervous system, and effector measures.

FIG. 2 illustrates a particular example of a system having an intelligent protocol generator and presenter device and individual mechanisms for intra-modality response synthesis.

FIG. 3 is one example of a sample flow process diagram showing a technique for obtaining neurological and neurophysiological data by Electroencephalography (EEG) triggered functional Magnetic Resonance Imaging (fMRI).

FIG. 4 illustrates particular examples of EEG response data that may be used to trigger fMRI.

FIG. 5 illustrates a particular example of an intra-modality synthesis mechanism for Electroencephalography (EEG).

FIG. 6 illustrates another particular example of synthesis for Electroencephalography (EEG).

FIG. 7 illustrates a particular example of a cross-modality synthesis mechanism.

FIG. 8 is one example of a sample flow process diagram showing a technique for obtaining neurological and neurophysiological data.

FIG. 9 illustrates a technique for addressing cross-modality interference.

FIG. 10 provides one example of a system that can be used to implement one or more mechanisms.

### DESCRIPTION OF PARTICULAR EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques and mechanisms of the present invention will be described in the context of EEG and fMRI. However, it should be noted that the techniques and mechanisms of the present invention apply to a variety of modality combinations, and not just EEG and fMRI. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

Overview

Neuro-response data including Electroencephalography (EEG) and Functional Magnetic Resonance Imaging (fMRI) data is collected, filtered and/or analyzed to evaluate the effectiveness of stimulus materials such as marketing and entertainment materials. A data collection mechanism obtains fMRI signals indicating a hemodynamic response to marketing or entertainment stimuli. In certain embodiments, such signals include region-specific blood oxygen level dependent (BOLD) signals that correlate with region-specific neural activity. fMRI signal acquisition is triggered by one or more EEG signatures indicating neural activity in response to exposure to stimulus materials.

Example Embodiments

Some efforts have been made to use isolated neurological and neurophysiological measurements to gauge subject responses. Some examples of central nervous system measurement mechanisms include Functional Magnetic Resonance Imaging (fMRI) and Electroencephalography (EEG). Autonomic nervous system measurement mechanisms include Galvanic Skin Response (GSR), Electrocardiograms (EKG), pupillary dilation, etc. Effector measurement mechanisms include Electrooculography (EOG), eye tracking, facial emotion encoding, reaction time etc.

EEG measures electrical activity associated with post synaptic currents occurring in the milliseconds range. Subcranial EEG can measure electrical activity with the most accuracy, as the bone and dermal layers weaken transmission of a wide range of frequencies. While surface EEG provides a wealth of electrophysiological information if analyzed properly, spatial resolution is poor.

fMRI measures blood oxygenation in the brain that correlates with increased neural activity. However, current implementations of fMRI have poor temporal resolution of a few seconds. Current implementations also rely on block design, in which magnetic resonance scans are continuously performed over a window of time to establish a steady-state BOLD response. Multiple individual responses within a window cannot be distinguished. Nevertheless, fMRI provides good spatial resolution of neural activity correlated with blood oxygenation.

Some conventional mechanisms of obtaining information about the effectiveness of various types of stimuli cite a particular neurological or neurophysiological measurement characteristic as indicating a particular thought, feeling, mental state, or ability. For example, one mechanism purports that the contraction of a particular facial muscle indicates the presence of a particular emotion. Others measure general activity in particular areas of the brain and suggest that activity in one portion may suggest lying while activity in another portion may suggest truthfulness. However, these mechanisms are severely limited in their ability to accurately reflect a subject's actual thoughts. It is recognized that a particular region of the brain can not be mapped to a particular thought. Similarly, a particular eye movement can not be mapped to a particular emotion. Even when there is a strong correlation between a particular measured characteristic and a thought, feeling, or mental state, the correlations are not perfect, leading to a large number of false positives and false negatives.

Consequently, the techniques and mechanisms of the present invention intelligently blend multiple modes such as EEG and fMRI to more accurately assess effectiveness of stimulus materials. According to various embodiments, manifestations of precognitive neural signatures are also blended with cognitive neural signatures and post cognitive neurophysiological manifestations to access the effectiveness of marketing and entertainment materials. In some examples, autonomic nervous system measures are themselves used to validate central nervous system measures. Effector and behavior responses are blended and combined with other measures.

Intra-modality measurement enhancements are made in addition to cross-modality measurement mechanism enhancements. According to various embodiments, brain activity is measured not just to determine the regions of activity, but to determine interactions and types of interactions between various regions. The techniques and mechanisms of the present invention recognize that interactions between neural regions support orchestrated and organized behavior. Thoughts and abilities are not merely based on one part of the brain but instead rely on network interactions between brain regions.

The techniques and mechanisms of the present invention further recognize that different frequency bands used for multi-regional communication can be indicative of the effectiveness of stimuli. For example, associating a name to a particular face may entail activity in communication pathways tuned to particular frequencies. According to various embodiments, select frequency bands are analyzed after filtering. The techniques and mechanisms of the present invention also recognize that high gamma band frequencies have significance. Inter-frequency coupling in the signals have also been determined to indicate effectiveness. Signals modulated on a carrier wave have also been determined to be important in evaluating thoughts and actions. In particular embodiments, the types of frequencies measured are subject and/or task specific. For example, particular types of frequencies in specific pathways are measured if a subject is being exposed to a new product.

The techniques and mechanisms of embodiments of the present invention further recognize that multi-regional activity and/or inter-regional communication, e.g., as measured by fMRI can be indicative of effectiveness of stimuli. For example, a particular emotion aroused by exposure to a stimulus may entail hemodynamic activity in a certain set of regions.

In particular embodiments, evaluations are calibrated to each subject and synchronized across subjects. In particular embodiments, templates are created for subjects to create a baseline for measuring pre and post stimulus differentials. According to various embodiments, stimulus generators are intelligent, and adaptively modify specific parameters such as exposure length and duration for each subject being analyzed.

Consequently, the techniques and mechanisms of the present invention provide a central nervous system, autonomic nervous system, and effector measurement and analysis system that can be applied to evaluate the effectiveness of materials such as marketing and entertainment materials. Marketing materials may include advertisements, commercials, media clips, brand messages, product brochures, company logos, etc. An intelligent stimulus generation mechanism intelligently adapts output for particular users and purposes. In addition to EEG and fMRI, a variety of modalities can be used including EKG, optical imaging, MEG, pupillary dilation, EOG, eye tracking, facial emotion encoding, reaction time, etc. Individual modalities such as EEG are enhanced by intelligently recognizing neural region communication pathways. Cross modality analysis is enhanced using a synthesis and analytical blending of central nervous system, autonomic nervous system, and effector signatures. Synthesis and analysis by mechanisms such as time and phase shifting, correlating, and validating intra-modal determinations allow generation of a composite output characterizing the effectiveness of various stimuli.

The techniques and mechanisms of the present invention contemplate performing multiple modality measurements simultaneously during a particular exposure to stimulus. For example, EEG and fMRI measurements are performed during exposure to a particular stimulus, with EEG triggering the fMRI data acquisition. The techniques and mechanisms of the present invention recognize that fMRI along with EEG and/or other mechanisms can be used to provide both higher temporal and spatial resolution for measurement of neurological activity. fMRI measures blood oxygenation levels. Blood flow increases to regions with increased neurological activity. However, the blood flow increase typically occurs several seconds after an event such as a stimulus event. Many systems perform continuous fMRI scans and are unable to isolate individual fMRI events. Consequently, the techniques and mechanisms of the present invention contemplate using EEG and/or other modalities to trigger fMRI in order to provide both improved spatial and temporal resolution for measurements of neurological responses from subjects exposed to marketing and entertainment materials.

In some examples, EEG brainwave signatures corresponding to particular stimulus events measured over thousands of trails are used to trigger fMRI. In other examples, event-related potentials (ERP) such as N1, P2, N2, and P3 peaks are used to trigger fMRI. According to various embodiments, ERP is a mechanism within the modality of EEG.

However, performing multiple modality measurements simultaneously presents its own set of problems. For example, one modality may interfere with the measurements from another modality. For example, EEG wires and electrodes may interfere with fMRI measurements. Consequently, filtering mechanisms are provided to address cross-modality interference, such as interference from EEG wires that disrupt fMRI measurements, or fMRI magnetic fields generating currents that alter EEG measurements. Filtered data is enhanced and combined to provide a blended effectiveness estimate of stimulus material effectiveness.

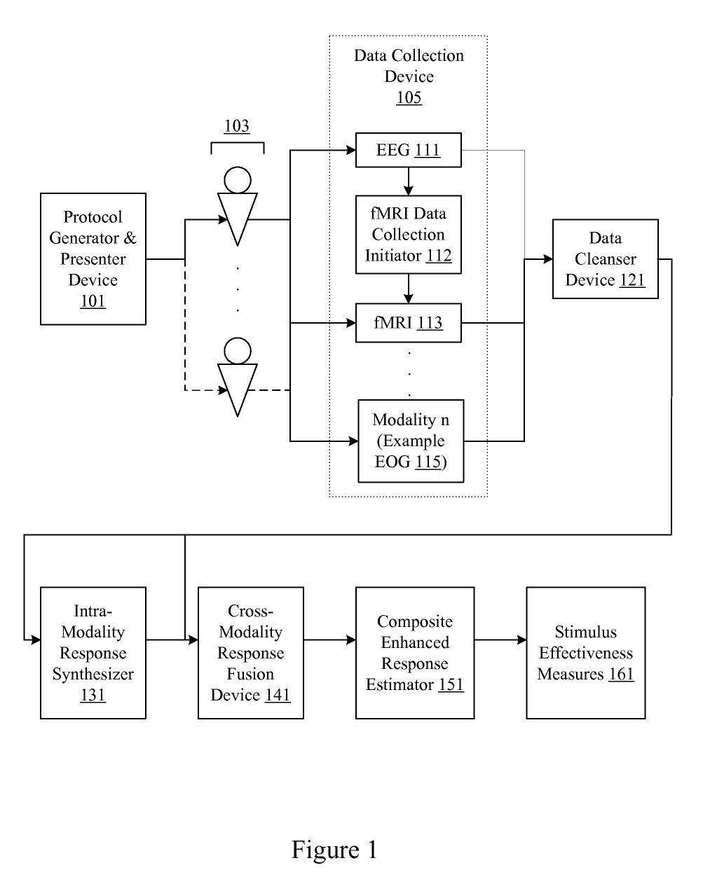

FIG. 1 illustrates one example of a system for determining the effectiveness of marketing and entertainment using EEG triggered fMRI. According to various embodiments, the neuro analysis system includes a protocol generator and presenter device 101. In particular embodiments, the protocol generator and presenter device 101 is merely a presenter device and merely presents stimuli to a user. The stimuli may be a media clip, a commercial, a brand image, a magazine advertisement, a movie, an audio presentation, particular tastes, smells, textures and/or sounds. The stimuli can involve a variety of senses and occur with or without human supervision. Continuous and discrete modes are supported. According to various embodiments, the protocol generator and presenter device 101 also has protocol generation capability to allow intelligent customization of stimuli provided to a subject.

According to various embodiments, the subjects 103 are connected to data collection devices 105 including EEG 111 and fMRI 113. In addition to EEG and fMRI, the data collection devices 105 may include a variety of neurological and neurophysiological measurement mechanisms such as EOG, GSR, EKG, pupillary dilation, eye tracking, facial emotion encoding, and reaction time devices, etc. In particular embodiments, the data collection devices 105 include EOG 115 in addition to EEG 111 and fMRI 113. In some instances, only EEG and fMRI devices are used. Data collection may proceed with or without human supervision.

The data collection device 105 collects neuro-physiological data from multiple sources. This includes a combination of devices such as central nervous system sources (EEG, fMRI), autonomic nervous system sources (GSR, EKG, pupillary dilation), and effector sources (EOG, eye tracking, facial emotion encoding, reaction time). In particular embodiments, data collected is digitally sampled and stored for later analysis. In particular embodiments, the data collected could be analyzed in real-time. According to particular embodiments, the digital sampling rates are adaptively chosen based on the neurophysiological and neurological data being measured.

In one particular embodiment, the neurological and neurophysiological analysis system includes EEG 111 measurements made using scalp level electrodes, fMRI 113 measurements made using a fMRI scanner and EOG 115 measurements through electrodes placed at specific locations on the face. Also in particular embodiments, the system also includes one or more of GSR measurements performed using a differential measurement system, a facial muscular measurement through shielded electrodes placed at specific locations on the face, and a facial affect graphic and video analyzer adaptively derived for each individual.

In particular embodiments, the system includes an fMRI data collection initiator 112. The fMRI data collection initiator 112 initiates acquisition of fMRI response data. In particular embodiments, fMRI data collection is triggered by one or more signals from EEG 111, e.g., that indicate a subject response to stimuli presented by protocol generator and presenter device 101. The fMRI data collection initiator identifies EEG response data or EEG signals indicating a response to the stimuli and initiates fMRI data collection. The fMRI data collection initiator 112 may include one or more devices each of which may be implemented using hardware, firmware, and/or software. It should be noted that although the fMRI data collection initiator 112 is shown located between EEG 111 and fMRI 113, the fMRI data collection initiator 112 like other components may have a location and functionality that varies based on system implementation. For example, some systems may initiate fMRI data collection using EEG response data that has been processed by one or more additional components of the system as described below.

In particular embodiments, the data collection devices are clock synchronized with a protocol generator and presenter device 101. The data collection system 105 can collect data from a single individual (1 system), or can be modified to collect synchronized data from multiple individuals (N+1 system). The N+1 system may include multiple individuals synchronously tested in isolation or in a group setting. In particular embodiments, the data collection devices also include a condition evaluation subsystem that provides auto triggers, alerts and status monitoring and visualization components that continuously monitor the status of the subject, data being collected, and the data collection instruments. The condition evaluation subsystem may also present visual alerts and automatically trigger remedial actions.

According to various embodiments, the neurological and neurophysiological analysis system also includes a data cleanser device 121. In particular embodiments, the data cleanser device 121 filters the collected data to remove noise, artifacts, and other irrelevant data using fixed and adaptive filtering, weighted averaging, advanced component extraction (like PCA, ICA), vector and component separation methods, etc. This device cleanses the data by removing both exogenous noise (where the source is outside the physiology of the subject) and endogenous artifacts (where the source could be neurophysiological like muscle movement, eye blinks, etc.).

The artifact removal subsystem includes mechanisms to selectively isolate and review the response data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (for example, an EEG nearest neighbor weighted averaging approach).

According to various embodiments, the data cleanser device 121 is implemented using hardware, firmware, and/or software. It should be noted that although a data cleanser device 121 is shown located after a data collection device 105 and before synthesis devices 131 and 141, the data cleanser device 121 like other components may have a location and functionality that varies based on system implementation. For example, some systems may not use any automated data cleanser device whatsoever. In other systems, data cleanser devices may be integrated into individual data collection devices.

The data cleanser device 121 passes data to the intra-modality response synthesizer 131. The intra-modality response synthesizer 131 is configured to customize and extract the independent neurological and neurophysiological parameters for each individual in each modality and blend the estimates within a modality analytically to elicit an enhanced response to the presented stimuli. In particular embodiments, the intra-modality response synthesizer also aggregates data from different subjects in a dataset.

According to various embodiments, the cross-modality response synthesis or fusion device 141 blends different intra-modality responses, including raw signals and signals output from synthesizer 131. The combination of signals enhances the measures of effectiveness within a modality. The cross-modality response fusion device 141 can also aggregate data from different subjects in a dataset.

According to various embodiments, the system also includes a composite enhanced response estimator (CERE) 151 that combines the enhanced responses and estimates from each modality to provide a blended estimate of the effectiveness of the marketing and entertainment stimuli for various purposes. Stimulus effectiveness measures are output at 161.

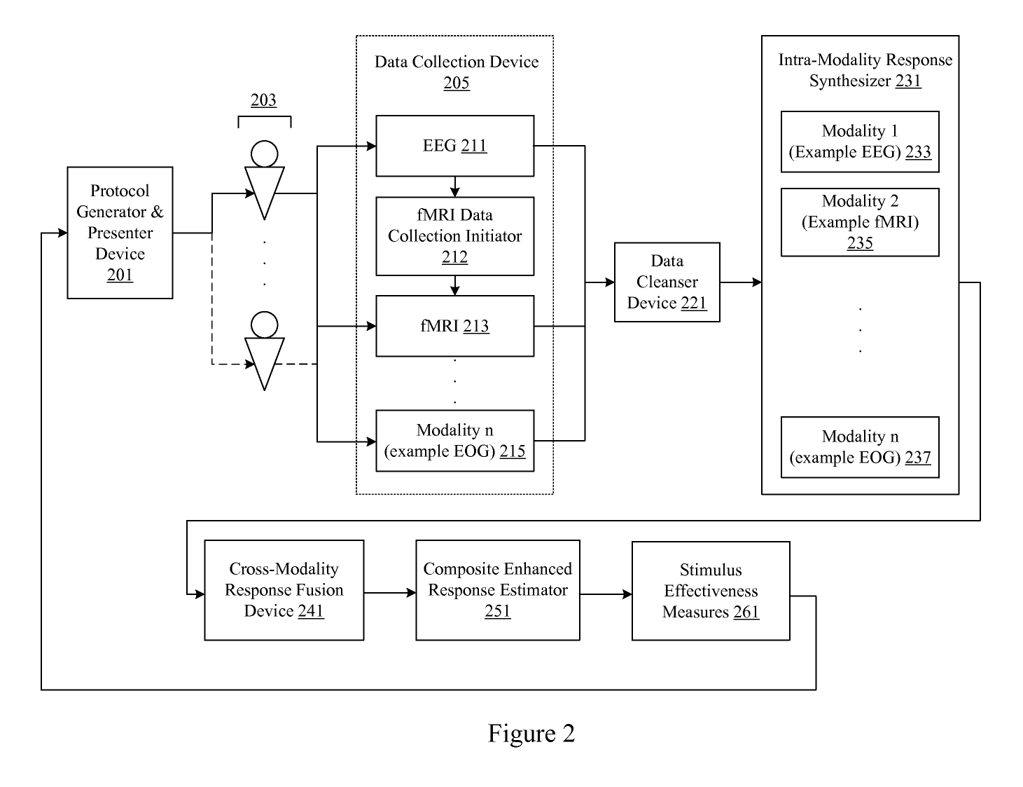

FIG. 2 illustrates a particular example of a system using EEG triggered fMRI and having an intelligent protocol generator and presenter device (where the intelligence could include a feedback based on prior responses) and individual mechanisms for intra-modality response synthesis.

According to various embodiments, the system includes a protocol generator and presenter device 201. In particular embodiments, the protocol generator and presenter device 201 is merely a presenter device and merely presents preconfigured stimuli to a user. The stimuli may be media clips, commercials, brand images, magazine advertisements, movies, audio presentations, particular tastes, textures, smells, and/or sounds. The stimuli can involve a variety of senses and occur with or without human supervision. Continuous and discrete modes are supported. According to various embodiments, the protocol generator and presenter device 201 also has protocol generation capability to allow intelligent modification of the types of stimuli provided to a subject. In particular embodiments, the protocol generator and presenter device 201 receives information about stimulus effectiveness measures from component 261.

The protocol generator and presenter device 201 dynamical adapts stimuli presentation by using information from the analysis of attention, analysis of emotional engagement, analysis of memory retention, analysis of overall visual, audio, other sensory effectiveness, and ad, show, or content effectiveness, implicit analysis of brand impact, implicit analysis of brand meaning, implicit analysis of brand archetype, implicit analysis of brand imagery, implicit analysis of brand words, explicit analysis of brand impact, explicit analysis of brand meaning, explicit analysis of brand archetype, explicit analysis of brand imagery, explicit analysis of brand words; analysis of characters in the ad, analysis of emotive response to characters in the ad/show/content, analysis of character interaction in the ad/show/content; elicitation of core components of the ad/show/content for print purposes, elicitation of core components of the ad/show/content for billboard purposes; elicitation of the ocular metrics like hot-zones in the ad/show/content by eye dwell time, micro and macro saccade separation, saccadic returns to points of interest; elicitation of points for product placement, elicitation of points for logo and brand placement; analysis of game effectiveness, analysis of product placement in games; analysis of website effectiveness, webpage dropoff in a site. According to various embodiments, the information is provided by component 261. In particular embodiments, the protocol generator and presenter device 201 can itself obtain some of this information

The protocol generator and presenter device 201 uses a data model along with linguistic and image tools like valence, arousal, meaning matched word/phrase generators, valence and arousal matched image/video selectors to generate parameters regarding the experiment. In particular examples, the protocol generator and presenter device 201 may vary individual presentation parameters like time and duration of the experiment, the number of repetitions of the stimuli based on signal to noise requirements, and the number and repetitions of the stimuli for habituation and wear-out studies, the type and number of neuro-physiological baselines, and the self reporting surveys to include.

In particular examples, the protocol generator and presenter device 201 customizes presentations to a group of subjects or to individual subjects. According to various embodiments, the subjects are connected to data collection devices 205. The data collection devices 205 may involve any type of neurological and neurophysiological mechanism such as EEG, fMRI, EOG, GSR, EKG, pupilary dilation, eye tracking, facial emotion encoding, reaction time, etc. In particular embodiments, the data collection devices 205 include EEG 211 and fMRI 213. In some instances, only two modalities, e.g., EEG and fMRI, are used. In other instances, additional modalities are used and may vary depending on the type of effectiveness evaluation. Data collection may proceed without or without human supervision.

The data collection device 205 automatically collects neuro-physiological data from multiple sources. This includes a combination of devices such as central nervous system sources (EEG, fMRI), autonomic nervous system sources (GSR, EKG, pupillary dilation), and effector sources (EOG, eye tracking, facial emotion encoding, reaction time). In particular embodiments, data collected is digitally sampled and stored for later analysis. The digital sampling rates are adaptively chosen based on the type of neurophysiological and neurological data being measured.

In particular embodiments, the system includes EEG 211 measurements made using scalp level electrodes, fMRI 213 measurements made using a fMRI scanner, EOG 215 measurements through electrodes placed at specific locations on the face, and a facial affect graphic and video analyzer adaptively derived for each individual.

In particular embodiments, the system includes an fMRI data collection initiator 212. The fMRI data collection initiator 212 initiates acquisition of fMRI response data. In particular embodiments, fMRI data collection is triggered by one or more signals from EEG 211, e.g., that indicate a subject response to stimuli presented by protocol generator and presenter device 201. The fMRI data collection initiator identifies EEG response data or EEG signals indicating a response to the stimuli and initiates fMRI data collection. The fMRI data collection initiator 212 may include one or more devices each of which may be implemented using hardware, firmware, and/or software. It should be noted that although the fMRI data collection initiator 212 is shown located between EEG 211 and fMRI 213, the fMRI data collection initiator 212 like other components may have a location and functionality that varies based on system implementation. For example, some systems may initiate fMRI data collection using EEG response data that has been processed by one or more additional components of the system as described below.

According to various embodiments, the data collection devices are clock synchronized with a protocol generator and presenter device 201. The data collection system 205 can collect data from a single individual (1 system), or can be modified to collect synchronized data from multiple individuals (N+1 system). The N+1 system could include multiple individuals synchronously recorded in a group setting or in isolation. In particular embodiments, the data collection devices also include a condition evaluation subsystem that provides auto triggers, alerts and status monitoring and visualization components that continuously monitor the status of the data being collected as well as the status of the data collection instruments themselves. The condition evaluation subsystem may also present visual alerts and automatically trigger remedial actions.

According to various embodiments, the system also includes a data cleanser device 221. In particular embodiments, the data cleanser device 221 filters the collected data to remove noise, artifacts, and other irrelevant data using fixed and adaptive filtering, weighted averaging, advanced component extraction (like PCA, ICA), vector and component separation methods, etc. This device cleanses the data by removing both exogenous noise (where the source is outside the physiology of the subject) and endogenous artifacts (where the source could be neurophysiological like muscle movement, eye blinks).

The artifact removal subsystem includes mechanisms to selectively isolate and review the output of each of the data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (for example, an EEG nearest neighbor weighted averaging approach), or removes these components from the signal.

According to various embodiments, the data cleanser device 221 is implemented using hardware, firmware, and/or software. It should be noted that although a data cleanser device 221 is shown located after a data collection device 205 and before synthesis devices 231 and 241, the data cleanser device 221 like other components may have a location and functionality that varies based on system implementation. For example, some systems may not use any automated data cleanser device whatsoever. In other systems, data cleanser devices may be integrated into individual data collection devices.

The data cleanser device 221 passes data to the intra-modality response synthesizer 231. The intra-modality response synthesizer is configured to customize and extract the independent neurological and neurophysiological parameters for each individual in each modality and blend the estimates within a modality analytically to elicit an enhanced response to the presented stimuli. In particular embodiments, the intra-modality response synthesizer also aggregates data from different subjects in a dataset. According to various embodiments, various modules perform synthesis in parallel or in series, and can operate on data directly output from a data cleanser device 221 or operate on data output from other modules. For example, EEG synthesis module 233 can operate on the output of fMRI synthesis module 235. EOG module 237 can operate on data output from EEG module 233.

According to various embodiments, the cross-modality response synthesis or fusion device 241 blends different intra-modality responses, including raw signals as well as signals output from synthesizer 231. The combination of signals enhances the measures of effectiveness within a modality. The cross-modality response fusion device 241 can also aggregate data from different subjects in a dataset.

According to various embodiments, the neuro analysis system also includes a composite enhanced response estimator (CERE) 251 that combines the enhanced responses and estimates from each modality to provide a blended estimate of the effectiveness of the marketing and advertising stimuli for various purposes. Stimulus effectiveness measures are output at 261. A portion or all of the effectiveness measures (intra-modality synthesizer, cross modality fusion device, and/or the CERE) can be provided as feedback to a protocol generator and presenter device 201 to further customize stimuli presented to users 203.

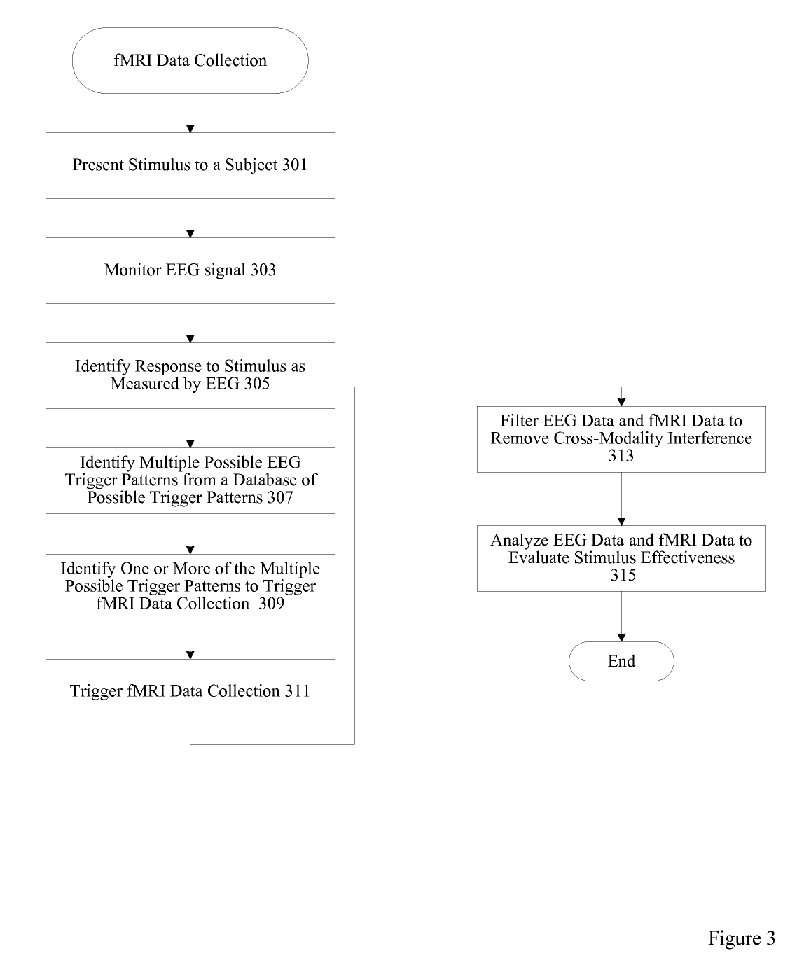

As indicated above, in particular embodiments the techniques and mechanisms of the present invention include collection of fMRI response data to measure stimulus effectiveness. FIG. 3 illustrates one technique for fMRI response data collection. At 301, a protocol and stimulus is provided to a subject. According to various embodiments, stimulus includes streaming video, media clips, printed materials, individual products, etc. The protocol determines the parameters surrounding the presentation of stimulus, such as the number of times shown, the duration of the exposure, sequence of exposure, segments of the stimulus to be shown, etc. Subjects may be isolated during exposure or may be presented materials in a group environment with or without supervision. At 303, EEG measurements indicating brain activity are monitored. According to various embodiments, data may be collected from scalp level electrodes. It should be noted that data may be collected from modalities such as ERP, EOG, GSR, etc., as well. At 305, one or more EEG signals indicating neural activity in response to the stimuli are identified. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making. Gamma waves occur between 30 and 60 Hz and are involved inbinding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment. However, the techniques and mechanisms of the present invention recognize that analyzing high gamma band (kappa-band: above 60 Hz) measurements, in addition to theta, alpha, beta, and low gamma band measurements. Particular sub-bands within each frequency range have particular prominence during certain activities. A subset of the frequencies in a particular band is referred to herein as a sub-band. For example, a sub-band may include the 40-45 Hz range within the gamma band. According to various embodiments, identifying a measurement indicating a response to a stimulus involves detecting a EEG signature such as a spike, polyspike or wave oscillations in one or more bands or sub-bands. In particular embodiments, identifying a measurement indicating a response to a stimulus involves recognition of one or more specific EEG signatures of brain activity.

According to various embodiments, real-time or near real-time identification of a measurement may be manual or automatic. For example, identification may be performed by visual inspection of an EEG trace or hardware or software-based EEG processing techniques. In particular embodiments, identification may involve various EEG analysis techniques including Fourier transforms and wavelet transforms.

In particular embodiments, identifying a response to stimulus to trigger fMRI involves identifying one or more EEG patterns. At 307, multiple possible trigger EEG patterns are identified from a database of multiple EEG trigger patterns. At 309, one or more of the identified possible trigger patterns is identified, e.g., by correlating measured response data to one or more possible EEG trigger patterns. The identified pattern or patterns is used to trigger fMRI data collection.

At 311, the onset of fMRI data collection is triggered by the identification of the EEG measurement indicating response to the stimulus. fMRI data collection involves acquisition of magnetic resonance images of the brain with a MRI scanner. Because the physiological response indicated by the fMRI signal may lag cortical activity, according to various embodiments, a lag period between a stimulus response as identified by EEG and fMRI data collection may be imposed. In other embodiments, fMRI image acquisition may occur immediately upon identification of stimulus response. Also according to various embodiments, initiating fMRI data collection may be manual or automatic.

According to various embodiments, fMRI measures change in blood oxygenation, regional cerebral blood flow, or regional cerebral blood volume. Changes in blood oxygenation and blood flow correlate with neural activity. In certain embodiments, a blood oxygen level dependent (BOLD) response is measured.

At 313, EEG and fMRI response data is filtered to remove cross-modality interference, such as such as interference from EEG wires that disrupt fMRI measurements and interference from fMRI magnetic fields generating currents that alter EEG measurements. At 315, filtered data is enhanced and combined to provide a blended effectiveness estimate of stimulus material effectiveness.

Using fMRI, block design and event-related responses of fMRI can be measured. Block design fMRI assumes that the BOLD response reaches steady state. The techniques and mechanisms of embodiments of the present invention recognize that a BOLD response is transient and may vary according to brain region as well as stimulus type and duration. Accordingly, in certain embodiments event-related fMRI (ER-fMRI) is performed. As described above, stimuli are presented according to a particular protocol. According to various embodiments, stimuli are presented in fixed, random or pseudorandom fashion for ER-fMRI. The protocol may also include time between stimulus onsets sufficient to allow recovery between consecutive stimuli.

A variety of analysis techniques may be performed to identify fMRI signatures of neural activity. These include model-based techniques including t-test, correlation analysis and general linear model (GLM) techniques as well as principle component analysis (PCA), independent component analysis (ICA) and clustering. According to various embodiments, fMRI response signatures or patterns, including spatial and temporal response signatures, are identified using these or other techniques. In certain embodiments, fMRI response signatures are correlated to neural activity associated with emotional engagement, attention and memory retention. For example, neural activity in the amygdala may be correlated with emotional and attention arousal in response to a stimulus. Multi-regional activity and/or inter-regional communication as measured by fMRI may also be correlated with attention, emotional engagement and memory. According to various embodiments, various spatial fMRI signatures are used to evaluate the effectiveness of stimuli.

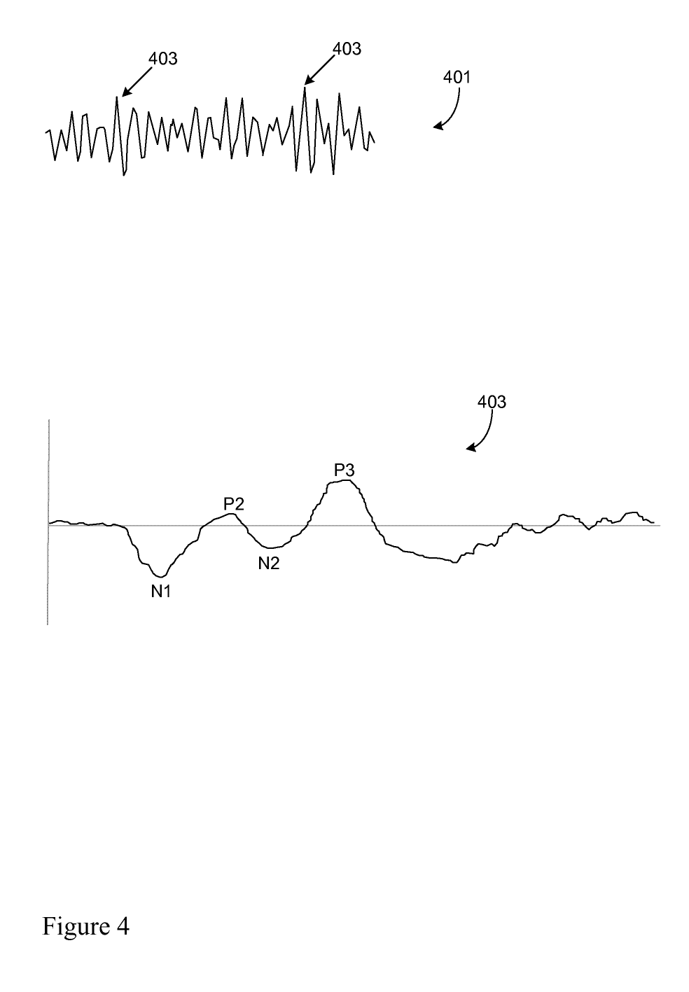

Examples of EEG response data that may be used to trigger fMRI data collection are shown in FIG. 4. At 401, an example of EEG trace data including peaks such as peak 403 is shown. One or more peaks or patterns of peaks, including EEG signatures, corresponding to stimulus response may be used to trigger fMRI. At 403, an example of ERPs including N1, P2, N2 and P3 peaks is shown. One or more peaks or patterns of peaks, including EEG signatures, corresponding to stimulus response may be used to trigger fMRI.

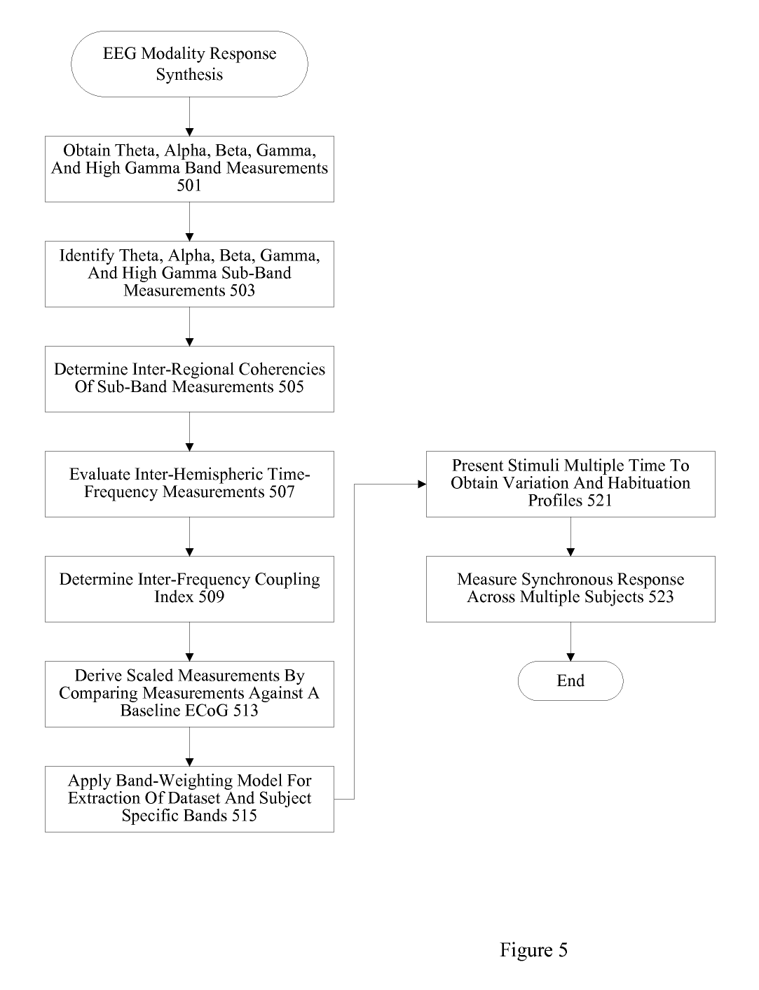

As indicated, in particular embodiments, an intra-modality synthesis mechanism is used to elicit an enhanced response to presented stimuli. FIG. 5 illustrates a particular example of an intra-modality synthesis mechanism. In particular embodiments, EEG response data is synthesized to provide an enhanced assessment of marketing and entertainment effectiveness. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making. Gamma waves occur between 30 and 60 Hz and are involved inbinding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment.

However, the techniques and mechanisms of the present invention recognize that analyzing high gamma band (kappa-band: Above 60 Hz) measurements, in addition to theta, alpha, beta, and low gamma band measurements, enhances neurological attention, emotional engagement and retention component estimates. In particular embodiments, EEG measurements including difficult to detect high gamma or kappa band measurements are obtained, enhanced, and evaluated at 501. At 503, subject and task specific signature sub-bands in the theta, alpha, beta, gamma and kappa bands are identified to provide enhanced response estimates. According to various embodiments, high gamma waves (kappa-band) above 80 Hz (typically detectable with sub-cranial EEG and magnetoencephalograophy) can be used in inverse model-based enhancement of the frequency responses to the stimuli.

Various embodiments of the present invention recognize that particular sub-bands within each frequency range have particular prominence during certain activities. A subset of the frequencies in a particular band is referred to herein as a sub-band. For example, a sub-band may include the 40-45 Hz range within the gamma band. In particular embodiments, multiple sub-bands within the different bands are selected while remaining frequencies are band pass filtered. In particular embodiments, multiple sub-band responses may be enhanced, while the remaining frequency responses may be attenuated.

At 505, inter-regional coherencies of the sub-band measurements are determined. According to various embodiments, inter-regional coherencies are determined using gain and phase coherences, Bayesian references, mutual information theoretic measures of independence and directionality, and Granger causality techniques of the EEG response in the different bands, as well as the power measures of response in fMRI and time-frequency response in EEG. In particular embodiments, inter-regional coherencies are determined using fuzzy logic to estimate effectiveness of the stimulus in evoking specific type of responses in individual subjects.

At 507, inter-hemispheric time-frequency measurements are evaluated. In particular embodiments, asymmetries in specific band powers, asymmetries in inter-regional intra-hemispheric coherences, and asymmetries in inter-regional intra-hemisphere inter-frequency coupling are analyzed to provide measures of emotional engagement.

At 509, inter-frequency coupling assessments of the response are determined. In particular embodiments, a coupling index corresponding to the measure of specific band activity in synchrony with the phase of other band activity is determined to ascertain the significance of the marketing and advertising stimulus or sub-sections thereof. At 513, a reference scalp over frequency curve is determined using a baseline electrocorticogram (ECoG) power by frequency function driven model. The reference scale power frequency curve is compared to an individual scalp record power by frequency curve to derive scaled estimates of marketing and entertainment effectiveness. According to various embodiments, scaled estimates are derived used fuzzy scaling.

At 515, an information theory based band-weighting model is used for adaptive extraction of selective dataset specific, subject specific, task specific bands to enhance the effectiveness measure. Adaptive extraction may be performed using fuzzy scaling. At 521, stimuli can be presented and enhanced measurements determined multiple times to determine the variation or habituation profiles across multiple presentations. Determining the variation and/or habituation profiles provides an enhanced assessment of the primary responses as well as the longevity (wear-out) of the marketing and entertainment stimuli. At 523, the synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

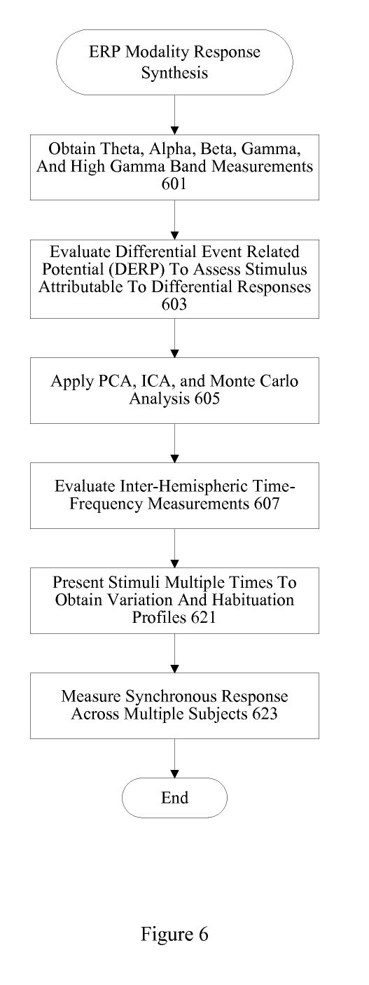

Although a variety of synthesis mechanisms are described, it should be recognized that any number of mechanisms can be applied—in sequence or in parallel with or without interaction between the mechanisms. In some examples, processes 521 and 523 can be applied to any modality. FIG. 6 illustrates a particular example of synthesis for Electroencephalography (EEG) data, including ERP and continuous EEG.

ERPs can be reliably measured using electroencephalography (EEG), a procedure that measures electrical activity of the brain. Although an EEG reflects thousands of simultaneously ongoing brain processes, the brain response to a certain stimulus may not be visible using EEG. ERP data includes cognitive neurophysiological responses that manifests after the stimulus is presented. In many instances, it is difficult to see an ERP after the presentation of a single stimulus. The most robust ERPs are seen after tens or hundreds of individual presentations are combined. This combination removes noise in the data and allows the voltage response to the stimulus to stand out more clearly. In addition to averaging, the embodiment includes techniques to extract single trial evoked information from the ongoing EEG. Using fMRI, block design and event-related responses of fMRI can be measured.

While evoked potentials reflect the processing of the physical stimulus, event-related potentials are caused by the “higher” processes, which might involve memory, expectation, attention, or changes in the mental state, among others. According to various embodiments, evidence of the occurrence or non-occurrence of specific time domain components in specific regions of the brain are used to measure subject responsiveness to specific stimulus.

According to various embodiments, ERP data and event-related responses can be enhanced using a variety of mechanisms. At 601, event related time-frequency analysis of stimulus response—event related power spectral perturbations (ERPSPs)—is performed across multiple frequency bands such as theta, delta, alpha, beta, gamma and high gamma (kappa). According to various embodiments, a baseline ERP is determined. At 603, a differential event related potential (DERP) is evaluated to assess stimulus attributable differential responses.

At 605, a variety of analysis techniques including principal component analysis (PCA), independent component analysis (ICA), and Monte Carlos analysis can be applied to evaluate an ordered ranking of the effectiveness across multiple stimuli. In particular embodiments, PCA is used to reduce multidimensional data sets to lower dimensions for analysis. ICA is typically used to separate multiple components in a signal. Monte Carlo relies on repeated random sampling to compute results. According to various embodiments, an ERP scenario is developed at 607 to determine a subject, session and task specific response baseline. The baseline can then be used to enhance the sensitivity of other ERP responses to the tested stimuli.

At 621, stimuli can be presented and enhanced measurements determined multiple times to determine the variation or habituation profiles across multiple presentations. Determining the variation and/or habituation profiles provides an enhanced assessment of the primary responses as well as the longevity (wear-out) of the marketing and entertainment stimuli. At 623, the synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

A variety of processes such as processes 621 and 623 can be applied to a number of modalities, including EOG, eye tracking, GSR, facial emotion encoding, etc. In particular embodiments, stimulus attributable differential fMRI responses are assessed and analyzed to evaluate an ordered ranking of the effectiveness across multiple stimuli. In some examples, evaluation of stimulus effectiveness recognizes that differential neural regional activation correlates to emotional responses, memory retention and engagement.

In addition, synthesis of data from mechanisms such as EOG and eye tracking can also benefit from the grouping objects of interest into temporally and spatially defined entities using micro and macro saccade patterns. Gaze, dwell, return of eye movements to primarily center around the defined entities of interest and inhibition of return to novel regions of the material being evaluated are measured to determine the degree of engagement and attention evoked by the stimulus.

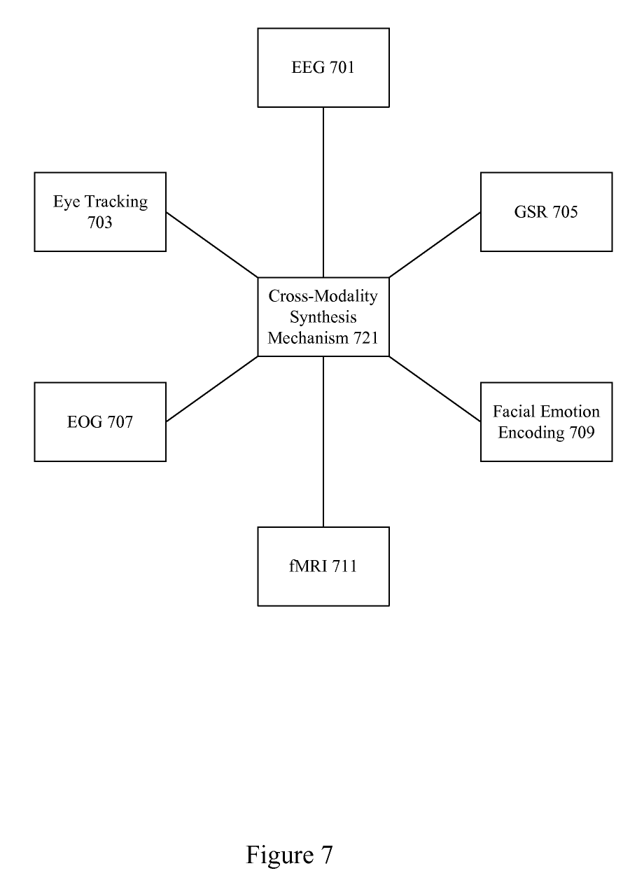

Although intra-modality synthesis mechanisms provide enhanced effectiveness data, additional cross-modality synthesis mechanisms can also be applied. FIG. 7 illustrates a particular example of a cross-modality synthesis mechanism 721. A variety of mechanisms such as EEG 701, Eye Tracking 703, GSR 705, EOG 707, facial emotion encoding 709, and fMRI 711 are connected to a cross-modality synthesis mechanism 721. Other mechanisms as well as variations and enhancements on existing mechanisms may also be included. According to various embodiments, data from a specific modality can be enhanced using data from one or more other modalities. In particular embodiments, EEG typically makes frequency measurements in different bands like alpha, beta and gamma to provide estimates of effectiveness. However, the techniques of the present invention recognize that effectiveness measures can be enhanced further using information from other modalities.

For example, facial emotion encoding measures can be used to enhance the valence of the EEG emotional engagement measure. EOG and eye tracking saccadic measures of object entities can be used to enhance the EEG estimates of effectiveness including but not limited to attention, emotional engagement, and memory retention. According to various embodiments, a cross-modality synthesis mechanism performs time and phase shifting of data to allow data from different modalities to align. In some examples, it is recognized that an EEG response will often occur hundreds of milliseconds before a facial emotion measurement changes. Correlations can be drawn and time and phase shifts made on an individual as well as a group basis. In other examples, saccadic eye movements may be determined as occurring before and after particular EEG responses. According to various embodiments, time corrected GSR measures are used to scale and enhance the EEG estimates of effectiveness including attention, emotional engagement and memory retention measures.

According to various embodiments, fMRI measures can be used to enhance EEG effectiveness measures. According to various embodiments, a cross-modality synthesis mechanism performs time and phase shifting of data to allow data from different modalities to align. In some examples, it is recognized that an EEG response will often occur several seconds before a hemodynamic response is measurable. Correlations can be drawn and time and phase shifts made on an individual as well as a group basis. In particular embodiments, data from fMRI and EEG is aligned using EEG triggered fMRI data collection information. In particular examples, it is recognized that spatial fMRI signatures correlate to attention, emotional engagement and memory retention. According to various embodiments, fMRI measures are used to scale and enhance the EEG estimates of effectiveness including attention, emotional engagement and memory retention measures.

Evidence of the occurrence or non-occurrence of specific time domain difference event-related potential components (like the DERP) in specific regions correlates with subject responsiveness to specific stimulus. According to various embodiments, ERP measures are enhanced using EEG time-frequency measures (ERPSP) in response to the presentation of the marketing and entertainment stimuli. Specific portions are extracted and isolated to identify ERP, DERP and ERPSP analyses to perform. In particular embodiments, an EEG frequency estimation of attention, emotion and memory retention (ERPSP) is used as a co-factor in enhancing the ERP, DERP and time-domain response analysis.

EOG measures saccades to determine the presence of attention to specific objects of stimulus. Eye tracking measures the subject's gaze path, location and dwell on specific objects of stimulus. According to various embodiments, EOG and eye tracking is enhanced by measuring the presence of lambda waves (a neurophysiological index of saccade effectiveness) in the ongoing EEG in the occipital and extra striate regions, triggered by the slope of saccade-onset to estimate the effectiveness of the EOG and eye tracking measures. In particular embodiments, specific EEG signatures of activity such as slow potential shifts and measures of coherence in time-frequency responses at the Frontal Eye Field (FEF) regions that preceded saccade-onset are measured to enhance the effectiveness of the saccadic activity data.

GSR typically measures the change in general arousal in response to stimulus presented. According to various embodiments, GSR is enhanced by correlating EEG/ERP responses and the GSR measurement to get an enhanced estimate of subject engagement. The GSR latency baselines are used in constructing a time-corrected GSR response to the stimulus. The time-corrected GSR response is co-factored with the EEG measures to enhance GSR effectiveness measures.

According to various embodiments, facial emotion encoding uses templates generated by measuring facial muscle positions and movements of individuals expressing various emotions prior to the testing session. These individual specific facial emotion encoding templates are matched with the individual responses to identify subject emotional response. In particular embodiments, these facial emotion encoding measurements are enhanced by evaluating inter-hemispherical asymmetries in EEG responses in specific frequency bands and measuring frequency band interactions. The techniques of the present invention recognize that not only are particular frequency bands significant in EEG responses, but particular frequency bands used for communication between particular areas of the brain are significant. Consequently, these EEG responses enhance the EMG, graphic and video based facial emotion identification.

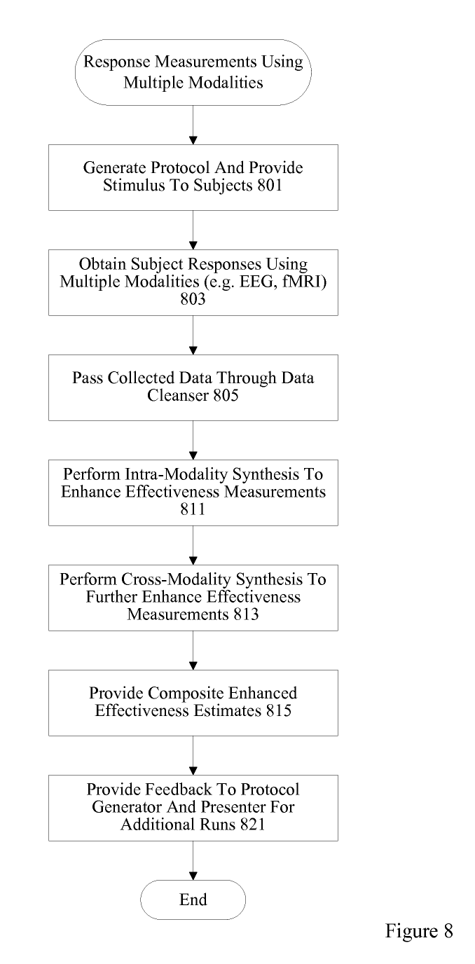

FIG. 8 is a flow process diagram showing a technique for obtaining neurological and neurophysiological data. At 801, a protocol is generated and stimulus is provided to one or more subjects. According to various embodiments, stimulus includes streaming video, media clips, printed materials, individual products, etc. The protocol determines the parameters surrounding the presentation of stimulus, such as the number of times shown, the duration of the exposure, sequence of exposure, segments of the stimulus to be shown, etc. Subjects may be isolated during exposure or may be presented materials in a group environment with or without supervision. At 803, subject responses are collected using a variety of modalities, such as EEG and fMRI. It should be noted that modalities such as ERP, EOG, GSR, etc., can be used as will. In some examples, verbal and written responses can also be collected and correlated with neurological and neurophysiological responses. At 805, data is passed through a data cleanser to remove noise and artifacts that may make data more difficult to interpret. According to various embodiments, the data cleanser removes EEG electrical activity associated with blinking and other endogenous/exogenous artifacts.

At 811, intra-modality response synthesis is performed to enhance effectiveness measures. At 813, cross-modality response synthesis is performed to further enhance effectiveness measures. It should be noted that in some particular instances, one type of synthesis may be performed without performing other types of synthesis. For example, cross-modality response synthesis may be performed with or without intra-modality synthesis. At 815, a composite enhanced response estimate is provided. At 821, feedback is provided to the protocol generator and presenter device for additional evaluations. This feedback may be provided by the cross-modality response synthesizer or by other mechanisms.

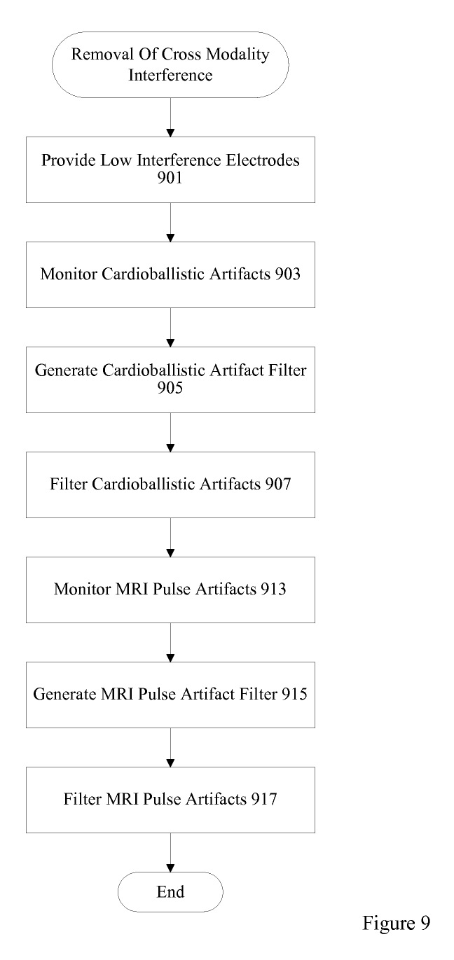

FIG. 9 illustrates one example of a technique for performing cross-modality interference filtering. Obtaining measurements using multiple modalities simultaneously address matters such as habituation and wear-out biases that occur when multiple modalities are used in sequence to measure subject responses. However, using multiple modalities simultaneously may lead to other inaccuracies. For example, electrodes used for EEG may interfere with fMRI measurements. Conventional silver, aluminum, and/or tin electrodes block radio frequency signals and prevent fMRI measurements in a substantial region beneath the electrode. Consequently, the techniques of the present invention provide minimal interference electrodes 901 that do not obstruct fMRI measurements as much as conventional electrodes. For example, sintered ceramic electrodes have leads that allow passage of radio frequency signals through the electrodes. The sintered ceramic electrodes do not block fMRI readings as much as conventional electrodes. EEG wires are also intelligently configured to prevent an antenna effect that absorbs radio frequency signals. In some embodiments, minimal wiring length is provided for the electrodes. Wiring may be twisted, shielded, etc. to minimize antenna effects. In some examples, electrodes may be connected with fiber optic cables or may be connected wirelessly to a receiving device or signal monitor to further reduce the amount of wiring.

According to various embodiments, fMRI magnetic fields can similarly introduce inaccuracies into EEG readings. In particular embodiments, cardioballistic artifacts are filtered. Cardioballistic artifacts are induced by head movements related to cardiac output. The head movements can generate current in the EEG wires when the EEG wires are located in strong magnetic fields such as fMRI induced magnetic fields. Cardioallistic artifacts are significant in comparison to EEG response measurements and can overshadow EEG response measurements. However, cadioballistic artifacts are regular. Consequently, the techniques of the present invention contemplate monitoring cardioballistic artifacts at 903, generating cardioballistic artifact filters at 905, and filtering cardioballistic artifacts at 907. According to various embodiments, cardioballistic artifact filters may be derived for groups or may be derived for individuals.

Pulse artifacts causing small movements in a strong magnetic field can similarly induce strong signals in EEG measurements. According to various embodiments, pulse artifacts are monitored at 913, pulse artifact filters are generated at 915, and pulse artifacts are filtered at 917.

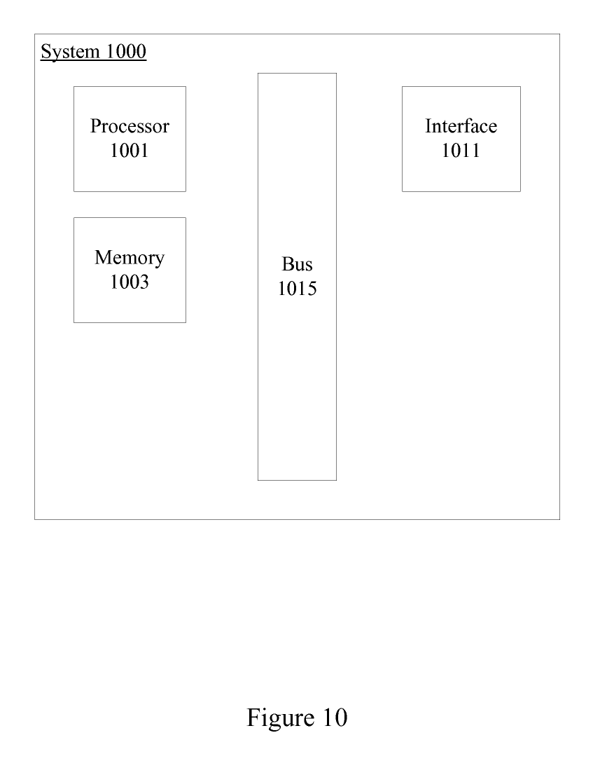

According to various embodiments, various mechanisms such as the data filtering mechanisms, the data collection mechanisms, the intra-modality synthesis mechanisms, cross-modality synthesis mechanisms, etc. are implemented on multiple devices. However, it is also possible that the various mechanisms are implemented in hardware, firmware, and/or software in a single system. FIG. 10 provides one example of a system that can be used to implement one or more mechanisms. For example, the system shown in FIG. 10 may be used to implement a data cleanser device or a cross-modality responses synthesis device.

According to particular example embodiments, a system 1000 suitable for implementing particular embodiments of the present invention includes a processor 1001, a memory 1003, an interface 1011, and a bus 1015 (e.g., a PCI bus). When acting under the control of appropriate software or firmware, the processor 1001 is responsible for such tasks such as pattern generation. Various specially configured devices can also be used in place of a processor 1001 or in addition to processor 1001. The complete implementation can also be done in custom hardware. The interface 1011 is typically configured to send and receive data packets or data segments over a network. Particular examples of interfaces the device supports include host bus adapter (HBA) interfaces, Ethernet interfaces, frame relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as data synthesis.

According to particular example embodiments, the system 1000 uses memory 1003 to store data, algorithms and program instructions. The program instructions may control the operation of an operating system and/or one or more applications, for example. The memory or memories may also be configured to store received data and process received data.

Because such information and program instructions may be employed to implement the systems/methods described herein, the present invention relates to tangible, machine readable media that include program instructions, state information, etc. for performing various operations described herein. Examples of machine-readable media include, but are not limited to, magnetic media such as hard disks, floppy disks, and magnetic tape; optical media such as CD-ROM disks and DVDs; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

Although the foregoing invention has been described in some detail for purposes of clarity of understanding, it will be apparent that certain changes and modifications may be practiced within the scope of the appended claims. Therefore, the present embodiments are to be considered as illustrative and not restrictive and the invention is not to be limited to the details given herein, but may be modified within the scope and equivalents of the appended claims.

What is claimed is:

1. A system, comprising:
a data collection mechanism including a plurality of modalities operable to obtain response data from a subject exposed to stimulus material including marketing and entertainment stimulus material, the response data comprising electroencephalography (EEG) response data and functional magnetic resonance imaging (fMRI) response data;
an fMRI data collection initiator operable to initiate fMRI data collection using an EEG signature included in the EEG response data;

2. The system of claim 1, wherein a filter connected to the data collection mechanism is operable to remove cross-modality interference from the EEG response data and the fMRI response data.

3. The system of claim 1, wherein a cross-modality response synthesizer is operable to analyze EEG response data and fMRI response data to evaluate effectiveness of the stimulus material, wherein EEG response data is combined with fMRI response data.

4. The system of claim 1 wherein the fMRI data collection initiator is operable to initiate fMRI data collection using an EEG spike.

5. The system of claim 1 wherein the fMRI data collection initiator is operable to initiate fMRI data collection using an EEG polyspike.

6. The system of claim 1 wherein the fMRI data collection initiator is operable to initiate fMRI data collection using a recognizable EEG pattern.

7. The system of claim 1, wherein the EEG signature comprises event related potential (ERP) data.

8. The system of claim 1, wherein removing cross-modality interference comprises removing EEG generated artifacts from fMRI response data and fMRI generated artifacts from EEG response data.

9. The system of claim 1, wherein EEG response data is aligned with fMRI response data, wherein aligning EEG response data with fMRI response data comprises time and phase shifting.

10. The system of claim 1, wherein fMRI measures are aligned and combined with electroencephalography (EEG) to enhance estimates of effectiveness.

11. The system of claim 1, wherein EEG response data is combined with fMRI response data to determine attention, emotional engagement, and memory retention.

12. A method, comprising:
obtaining response data using a plurality of modalities, the response data obtained from a subject exposed to stimulus material including marketing and entertainment stimulus material, the response data comprising electroencephalography (EEG) response data and functional magnetic resonance imaging (fMRI) response data, wherein obtaining response data using a plurality of modalities comprises triggering fMRI response data collection using an EEG signature.

13. The method of claim 12, further comprising removing cross-modality interference from the EEG response data and the fMRI response data.

14. The method of claim 12, further comprising analyzing EEG response data and fMRI response data to evaluate effectiveness of the stimulus material, wherein EEG response data is combined with fMRI response data.

15. The method of claim 13, wherein removing cross-modality interference comprises removing EEG generated artifacts from fMRI response data and fMRI generated artifacts from EEG response data.

16. The method of claim 12, wherein EEG response data is aligned with fMRI response data, wherein aligning EEG response data with fMRI response data comprises time and phase shifting.

17. The method of claim 12, wherein the EEG signature comprises event related potential (ERP) data.

18. The method of claim 12, wherein triggering fMRI response data collection comprises identifying an EEG spike.

19. The method of claim 12, wherein triggering fMRI response data collection comprises identifying an EEG polyspike.

20. An apparatus, comprising:
means for obtaining response data using a plurality of modalities, the response data obtained from a subject exposed to stimulus material including marketing and entertainment stimulus material, the response data comprising electroencephalography (EEG) response data and functional magnetic resonance imaging (fMRI) response data;
means for triggering fMRI data collection using EEG response data;
means for removing cross-modality interference from the EEG response data and the fMRI response data;
means for analyzing EEG response data and fMRI response data to evaluate effectiveness of the stimulus material, wherein EEG response data is combined with fMRI response data.
